(19) United States
(12) Patent Application Publication
(10) Pub. No.: US 2009/0145444 A1
(43) Pub. Date: Jun. 11, 2009

(54) DEVICES, SYSTEMS AND METHODS RELATING TO DUST MASKS HAVING EAR PROTECTION

(76) Inventor: Thomas William Edwards, Everett, WA (US)

Correspondence Address: GRAYBEAL JACKSON LLP, 155 - 108TH AVENUE NE, SUITE 350, BELLEVUE, WA, 98004-5973, US

(21) Appl. No.: 12/323,853

(22) Filed: Nov. 26, 2008

Related U.S. Application Data
(60) Provisional application No. 61/004,884, filed on Nov. 30, 2007.
(60) Provisional application No. 61/032,002, filed on Feb. 27, 2008.
(60) Provisional application No. 61/036,440, filed on Mar. 13, 2008.

(51) Int. Cl. A62B 23/02; A41D 13/11; A61F 9/02

(52) U.S. Cl. 128/858; 128/863; 128/206.13

(57) ABSTRACT

A dust mask, the dust mask being lightweight, disposable and configured for a human face, the dust mask further being substantially continuous, and unitary if desired, and sized and configured to extend from ear to ear and to cover all of and substantially only a mouth, nose and ears, the mask further configured to transmit air inhaled by or exhaled by the user yet substantially completely block at least dust from reaching the nose, mouth and ears of the user. The mask can be further configured to substantially completely block at least one undesired air-borne contaminant in addition to dust and can comprise a central portion covering the user's nose and mouth and two earpieces seamlessly attached to the central portion. The mask can further comprise a pliable, resilient nose bridge that can be adjusted to conform to the nose of the user as well as a variety of other structural elements and configurations.

### CROSS-REFERENCE TO RELATED APPLICATIONS

The present application claims the benefit of copending U.S. Provisional Patent Application Ser. No. 61/004,884, filed Nov. 30, 2007; the present application also claims the benefit of copending U.S. Provisional Patent Application Ser. No. 61/032,002, filed Feb. 27, 2008; the present application also claims the benefit of copending U.S. Provisional Patent Application Ser. No. 61/036,440, filed Mar. 13, 2008; all of the foregoing applications are incorporated herein by reference in their entireties and for all their teachings and disclosures.

### BACKGROUND

Dust masks are well-known devices that are typically used in industrial, medical or other situations to protect a user from inhaling unwanted air-borne particles such as drywall dust, paint molecules, viruses, etc. Lightweight dust masks are also well-known, and are configured to cover the nose and mouth of a user, typically held to the user's head by an elastic band. FIG. 6 depicts such a mask 102 on the head of user 104. The mask 102 comprises relatively thin elastic straps 108 and does not cover the ears 110 of the user. Such masks, therefore, leave the ears vulnerable to the unwanted particles, are uncomfortable to wear, may not remain in a desired position on the user's face, and otherwise do not protect the user as effectively or comfortably as may be desired.

Thus, there has gone unmet a need for improved dust mask devices, systems, methods, etc., that protect the ears, stay in place better, and/or are more comfortable. The present systems and methods, etc., provide one or more of these and/or other advantages.

### SUMMARY

The present devices, systems, methods, etc., provide lightweight dust masks that cover all of the mouth, nose and ears of the user, thereby protecting the user's hearing and keeping the ears clean without restricting the hearing ability (unless sound protection is also desired, in which case sound reducing materials and/or configurations can be provided). The masks are substantially continuous from ear to ear and can be a simple unitary design (i.e., the mask is not only continuous from ear to ear but at least one ply of the layers of material making up the mask extends from ear to ear without a break). The masks typically comprise multiple layers of protective material plus one or more straps, resilient ear “hooks” or other retaining devices to comfortably and securely hold the mask in place. The mask can also comprise, if desired, an exhalation vent and rigidity elements such as a nose bridge.

The masks can be made out of the same materials that prior dust masks are made from, and/or of other materials such as activated carbon for use with different chemicals and particles.

One aspect herein comprises a lightweight, disposable dust mask configured for a human face, the dust mask comprising a substantially continuous body sized and configured to extend from ear to ear and to cover all of and substantially only a mouth, nose and ears of a user, the mask further configured to transmit air inhaled by or exhaled by the user yet substantially completely block at least dust from reaching the nose, mouth and ears of the user.

In some embodiments, the mask can be further configured to substantially completely block at least one undesired air-borne contaminant in addition to dust, can comprise a central portion covering the user's nose and mouth and two earpieces seamlessly attached to the central portion, and can comprise a pliable, resilient nose bridge that can be adjusted to conform to the nose of the user; the mask further can comprise a softening element such as a foam piece under the nose bridge. The mask further can comprise at least one retaining device configured to hold the mask in place on the user, which can comprise at least one strap extending from earpiece to earpiece of the mask or at least two resilient ear hooks configured to at least partially loop around the ears of the user to hold the mask in place via mechanical interaction with the ears. The retaining device can be the at least one strap extending from earpiece to earpiece and extending from at least one of a top or back edge of each earpiece such that the strap does not contact the ears of the user. The at least one strap can comprise at least one of an elastic band or two opposed partial straps joining at mating male and female sides of a hook-and-loop connector fabric.

The mask can comprise multiple layers of protective material extending from ear to ear, and can comprise at least one layer of protective material extending from ear to ear and at least one layer of protective material covering substantially only one or both of the mouth and nose. The mask can also comprise at least one layer of protective material extending from ear to ear and at least one, non-continuous layer of protective material comprising at least two separate ear portions, each ear portion covering substantially only one ear. The ear portions can comprise at least one sound reducing material sufficient to protect the ears of a user from industrial levels of ambient sound, which can be at least one of an industrial spray painting machine, a cutting tool or an internal combustion engine.

The mask can be an industrial dust mask configured to protect a user from at least one air-borne particulate. The air-borne particulate can be at least one of auto paint, asbestos, a hazardous chemical, pollen or fiberglass dust. The mask can be a medical mask configured to protect a user from at least one air-borne medically undesirable element and the air-borne medically undesirable element can be at least one of blood splatter, an infectious organism or a hazardous chemical.

The mask can be made of at least one of cloth, plastic or paper products, and substantially all of the mask can be porous, and the mask can comprise at least one inhalation/exhalation vent. A lower contour of the dust mask can extend from behind the ears, along the jaw line of the user then to and/or under the chin; an upper contour of the mask extends from the ears, along the cheeks and then over the bridge of the nose. The upper contour of the mask can be specifically conformed to match a lower margin of corresponding specific eye protection for the user.

The mask further can comprise at least one of a resilient, pliable chin piece, ribbing, or strut that can be configured to cause the mask to conform very closely to the user or to create a desired, defined space between the mask and the user at least one desired point such as at the chin or the ears. The space can be configured to fit at least one sound deadening material disposed between an outer wall of the mask and the user's ears. The space can be configured to fit an ear muff or a respirator. The mask further can comprise a liner around substantially an entirety of an edge of the entire mask, the liner configured for improved strength and/or fit relative to the user.

An outer wall of the mask further can comprise at least one artistic design such as a skull and bones or a smile.

In a further aspect, the mask can be an industrial dust mask, the dust mask being lightweight, disposable and configured for a human face, the dust mask comprising a substantially unitary, substantially continuous body sized and configured to extend from ear to ear of a user and sized and configured to cover all of and substantially only a mouth, nose and ears of a user, the mask further configured to transmit air inhaled by or exhaled by the user yet substantially completely block at least one air-borne particulate from reaching the nose, mouth and ears of the user, the air-borne particulate can be at least one of dust, auto paint, a hazardous chemical or fiberglass dust.

The mask can also be a medical dust mask, the dust mask being lightweight, disposable and configured for a human face, the dust mask further being substantially unitary and continuous from ear to ear and sized and configured to cover all of and substantially only a mouth, nose and ears of a user, the mask further configured to transmit air inhaled by or exhaled by the user yet substantially completely block at least one air-borne medically undesirable element from reaching the nose, mouth and ears of the user, the air-borne particulate can be at least one of blood splatter, an infectious organism or a hazardous chemical.

In still a further aspect, the mask can be part of system comprising at least one of the masks with point of sale packaging; in certain embodiments, the system comprises multiple dust masks, for example 2, 3 6 or 10, stacked on each other such that the masks are nestled one inside another, if desired without intervening material, and comprising point of sale packaging.

The system can also comprise a glasses or goggles configured for the human user. An upper contour of the dust mask and a lower contour of the glasses or goggles can be configured to provide a substantially complementary fit to each other.

Still a further aspect comprises methods of making a dust mask comprising: manufacturing a dust mask as described above and elsewhere herein. The method further can comprise placing multiple masks into a point of sale package.

The methods can also comprise using a dust mask as discussed herein, wherein the methods can comprise applying the dust mask to the face and ears of a user to cover the face and ears of the user, removing the dust mask from the user and disposing of the dust mask. The dust mask can be a first dust mask and the methods further can comprise, after removing the first dust mask, applying a second dust mask substantially similar to the first dust mask on the user to cover the face and ears of the user. The applying can comprise retrieving the second dust mask from a stack of dust masks as discussed herein wherein there is no intervening material between the masks. The methods further can comprise substantially preventing at least one undesired air-borne particulate from reaching the nose, mouth and ears of the user. The methods further can comprise substantially preventing undesired noise from reaching the ears of the user.

In still yet another aspect, the lightweight dust mask can be substantially continuous and sized and configured to cover at least a mouth and nose of a user, the mask further configured to transmit air inhaled by or exhaled by the user yet substantially completely block at least unwanted air-borne particles from reaching at least the nose and mouth of the user, the mask further can comprise at least a first retaining strap extending between opposed substantially upper lateral edges of the mask and configured such that the first retaining strap extends substantially behind the head of the user and a second retaining strap extending between or about the opposed substantially upper lateral edges and configured such that the second retaining strap extends substantially over the head of the user. The dust mask can further cover the ears of the user as discussed elsewhere herein. The first strap can be attached along the ultimate upper edge of the mask.

These and other aspects, features and embodiments are set forth within this application, including the following Detailed Description and attached drawings. Unless expressly stated otherwise, all embodiments, aspects, features, etc., can be mixed and matched, combined and permuted in any desired manner.

### BRIEF DESCRIPTION OF THE DRAWINGS

FIG. 1 depicts a side plan view of a dust mask comprising ear protection as discussed herein.

FIG. 2 depicts a side plan view of a further embodiment of a dust mask comprising ear protection.

FIG. 3 depicts a top plan view of a dust mask comprising ear protection.

FIG. 4 depicts a side plan view of an embodiment of the dust masks herein wherein the dust mask comprises resilient ear hooks without straps.

FIG. 5 depicts a rear plan view of an alternative strap configuration.

FIG. 6 depicts, in side silhouette, a view of a prior art dust mask in place on the head of a user.

FIG. 7 depicts, in side silhouette, a view of a dust mask as discussed herein in place on the head of a user.

FIG. 8 depicts, in silhouette, a view of a mask in place on the head of a user with eye protection also on the head of the user.

FIG. 9 depicts an embodiment of the masks herein wherein the front of the mask comprises a desired artistic design.

FIG. 10 depicts an embodiment of the masks herein wherein the front of the mask comprises a desired artistic design.

FIG. 11 depicts an embodiment of the masks herein wherein the front of the mask comprises a desired artistic design.

FIG. 12 depicts an embodiment of the masks herein wherein the front of the mask comprises a desired artistic design.

### DETAILED DESCRIPTION

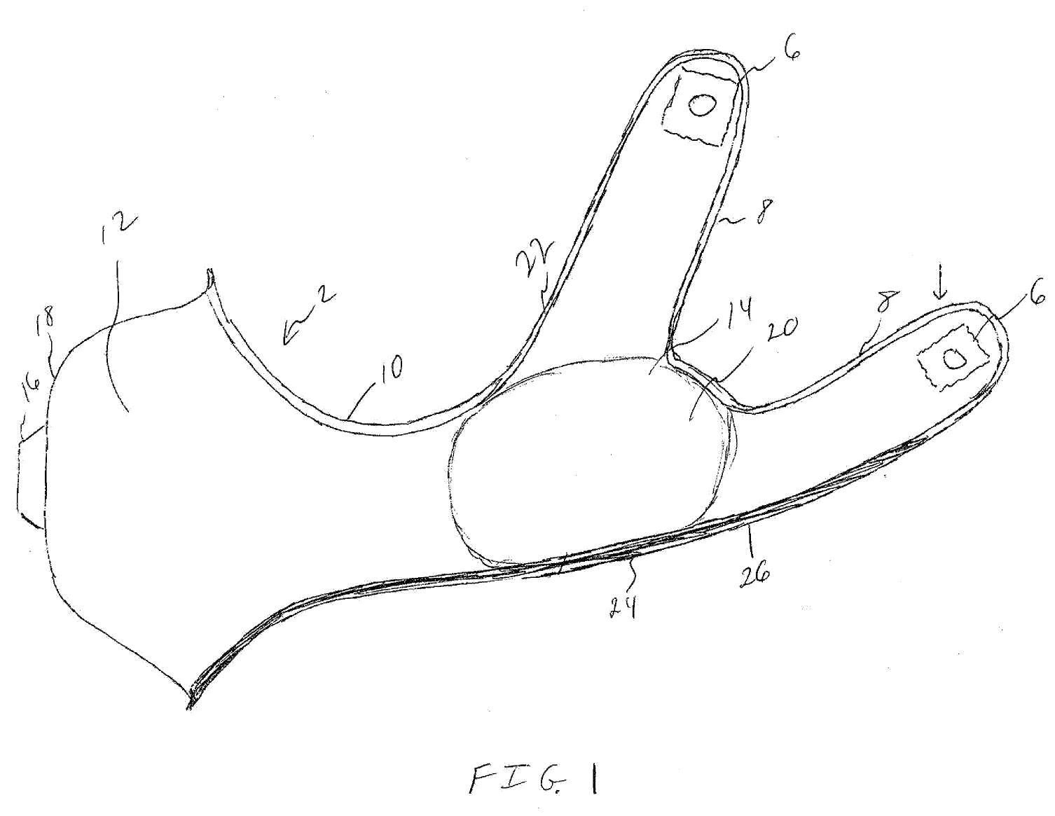

Turning first to the Figures for a discussion of the exemplary embodiments shown therein, FIG. 1 depicts a side view of a dust mask as discussed herein wherein mask 2 comprises a body 10 comprising a front piece 12 configured to cover the nose and mouth of the user and an earpiece 14 configured to cover the ears of a user (only one of the earpieces 14 is shown in FIG. 1). The body 10 of the mask 2 is substantially contiguous and sized and configured to extend from ear to ear and to substantially cover all of and only the mouth, nose and ears of the user. Typically, the mask 2 is lightweight and disposable so that it is relatively inexpensive, and can be provided in easily stackable, easily packable groups so that a user can use one until it becomes fouled by air-borne particles such as dust, paint spray, medical detritus, etc., then the user can easily remove the mask, throw it away into an appropriate receptacle, and then replace the mask with a readily available replacement mask.

The materials comprising the mask can be any appropriate materials and are typically selected for the particular use for which the mask is intended. For example, in an industrial situation, such as a manufacturing plant or an auto body painting or fiber glass shop, the mask is typically made of paper or plastic and configured to filter out air-borne particles of a certain size that is likely to be found in that environment (for example, paint droplets or molecules, fiber glass particles, or dust from materials such as metal, wood, glass, stone, etc., that are being machined). In the situation where the mask is to be used for medical situations such as surgery or immuno-compromised care, or certain high-tech situations such as clean rooms where absolute cleanliness and/or sterility are important, the mask is made of sterilized and/or completely non-dust-emitting materials so that the mask does not introduce any unwanted pathogens, contaminants, etc., into the operating environment.

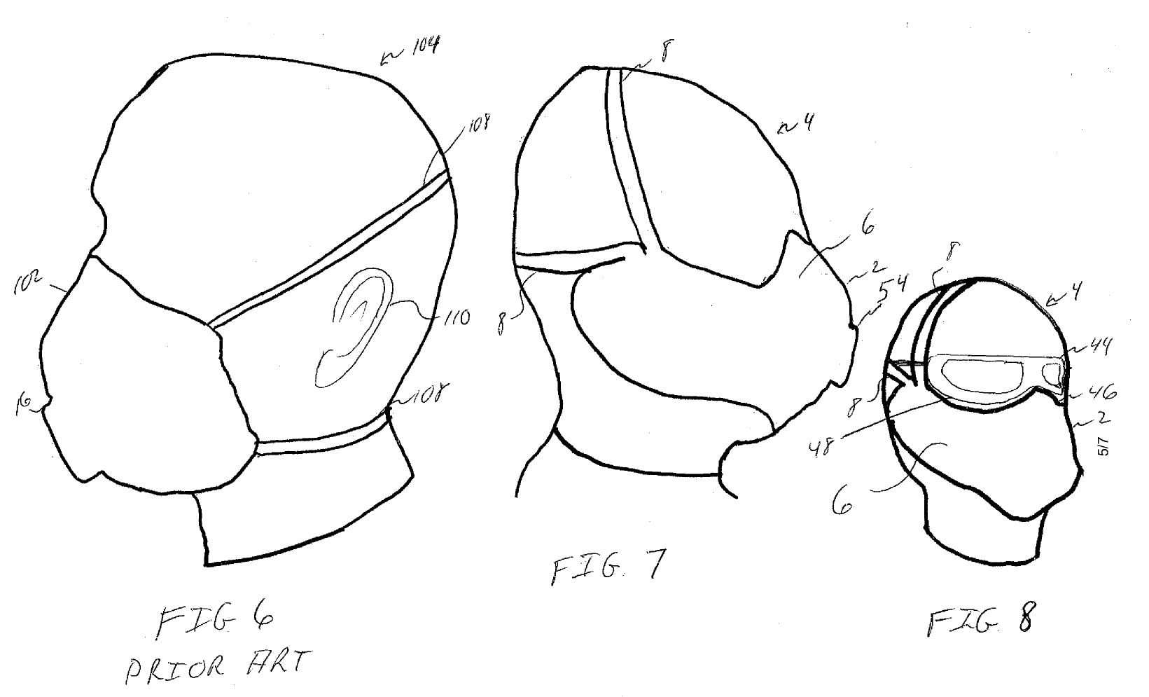

Still referring to the exemplary embodiment in FIG. 1, the mask also comprises at least one retaining device configured to hold the mask in place on the user. In the embodiment of FIG. 1, the retaining device is two straps 8, each contains a snap 6 with the opposing straps or retention locations on the other side of the mask comprising corresponding snaps to receive snaps 6 and securely hold mask 2 to the head of the user (the head of the user is not shown in FIG. 1 is but shown, for example, as user's head 4 in FIGS. 7 and 8). The mask 2 can, if desired, include an exhalation vent 16 which is typically located at substantially the front 18 of the mask where the user's nose and mouth are located. Earpiece 14 can comprise an ear bulge 20 so that it more comfortably fits around the user's ear. If desired, the earpiece 14 can further comprise materials that are particularly suited for the ear, such as soft materials that do not irritate the ear, sound deadening materials to protect the ear from ambient noise such as industrial levels of noise such as jackhammers, milling machines, high pressure paint sprayers, etc. Such materials can be incorporated into ear bulge 20 advantageously, although they can also be used without a bulge for the ears.

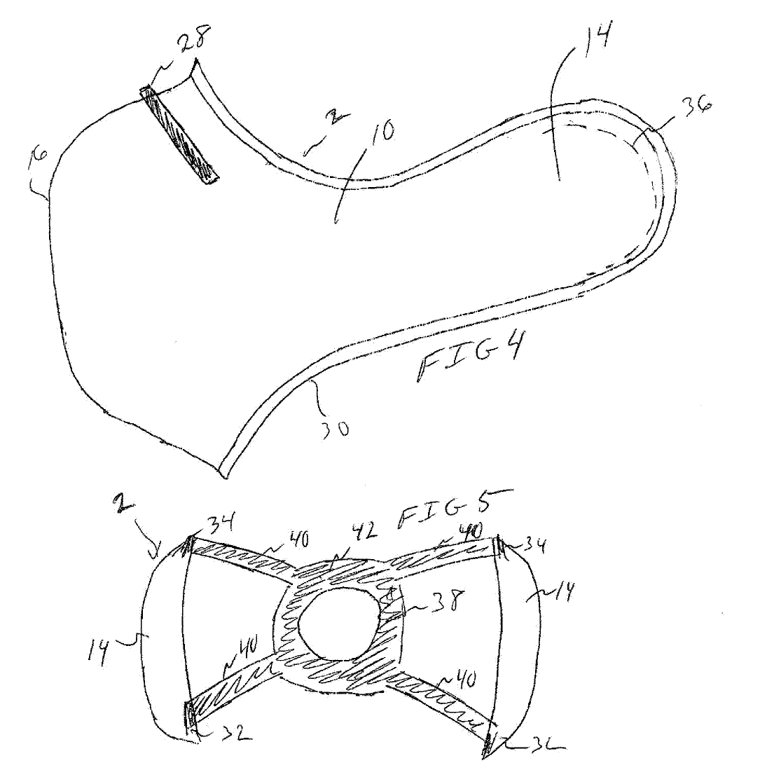

As depicted in FIG. 1, in certain embodiments the retaining devices are thick straps 8 which can be made of the same material as the dust mask or of a different material. Thus, either or both the ear pieces or substantially the entire mask can be made of an elastomeric material that stretches to adapt to the particular configuration of the individual user's head. By providing wider straps than the typical thin rubber bands depicted in FIG. 6 showing the prior art, the straps can be more comfortable and also hold the mask more securely in place on the user's head, thereby reducing both discomfort and the likelihood that the mask will slip or not fit properly thereby permitting unwanted air-borne particles to enter the user's ears, nose and mouth.

As depicted in exemplary embodiment in FIG. 1, the periphery of the mask can be ridged, thickened or comprise an additional material such as an elastomeric material (typically wrapped within a material more comfortable for the user) to provide a peripheral ridge 24 that extends along all or a desired portion of the mask 2. This ridge can be configured to aid, for example, in improved comfort for the user by providing a more snug fit that conforms to the individual characteristics of the individual face of the user, as well as improved safety characteristics by providing an improved fit to the user. If desired, the periphery can comprise different materials or characteristics at different locations, for example, the lower edge 26 of the mask may be elastomeric while the upper edge may provide a thickened, rigid portion.

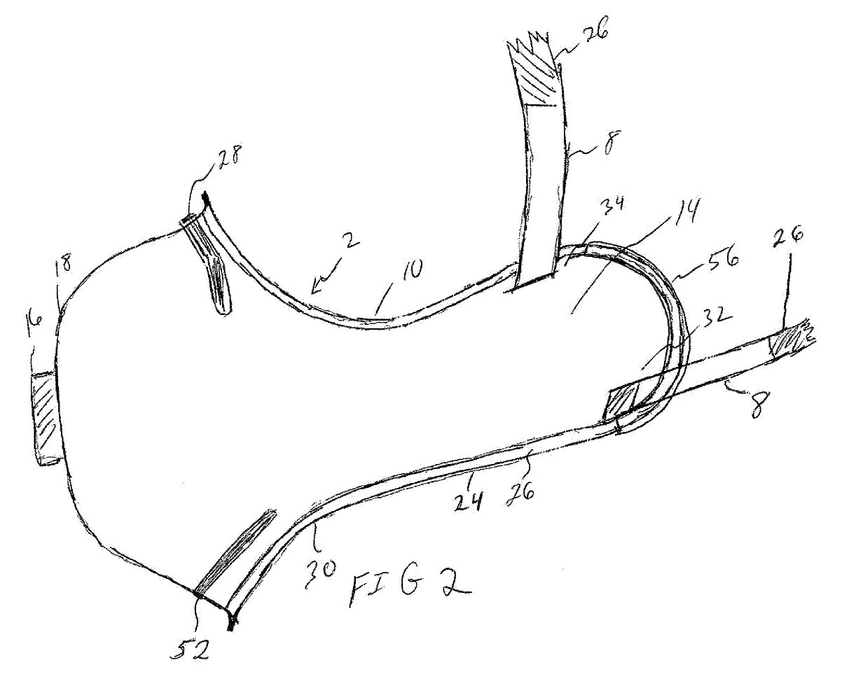

FIG. 2 depicts an additional exemplary embodiment of a dust mask 2. The dust mask in FIG. 2 is similar to the dust mask in FIG. 1 except the straps 8 comprise a hook and loop connector fabric such as VELCRO® on the ends of the straps. Mating hook and loop connector fabric pieces are on the opposing straps or opposing side of the mask or other suitable location, so that there are mating male and female sides of a hook and loop connector fabric configured such that the straps can be easily passed around the head of a user and lengthened or shortened to fit the particular head of the user. As with the embodiment in FIG. 1, the bottom periphery or lower contour 30 snugly and firmly fits under the chin of the user then trails up the neck to surround and cover the complete ear of the user. In the embodiment in FIG. 2, the lower strap departs from a lower lateral region 32 while a second, upper strap 26 departs from an upper lateral edge 34 of the body 10 of mask 2. Further, the embodiment in FIG. 2 comprises a resilient nose bridge 28 and a resilient nose chin piece 52.

Mask 2 in FIG. 2 further comprises at the front 18 of mask 2 an exhalation vent 16. Such vents are well-known and are configured to ease and/or improve the inhalation and exhalation by the user, and/or to improve filtration of unwanted particles if desired. For example, while the entire body 10 of mask 2 can be porous, if desired only the exhalation vent 16 can be porous while the remainder of body 10 can be either porous or substantially or completely impermeable. Further, if desired, exhalation vent 16 can comprise one or more exchangeable filters or filter elements such as activated carbon cartridges, etc.

FIG. 3 depicts a top view of a mask as discussed herein wherein the mask, as also depicted in FIG. 2, comprises a resilient nose bridge 28. The resilient nose bridge can, if desired, be deformable, for example via the use of metal, so that it can be adjusted by the user to configure to the individual shape of the individual user's nose and face. Conversely and/or in addition, the resilient nose bridge can provide structure to the mouth and nose area of the mask so that the bulge about the user's nose and mouth is maintained even under relatively difficult conditions such as high wind or pressure or anticipated external contact. In the view in FIG. 3, the ear bulge 20 of earpieces 14 can be more readily seen than in some of the other figures herein.

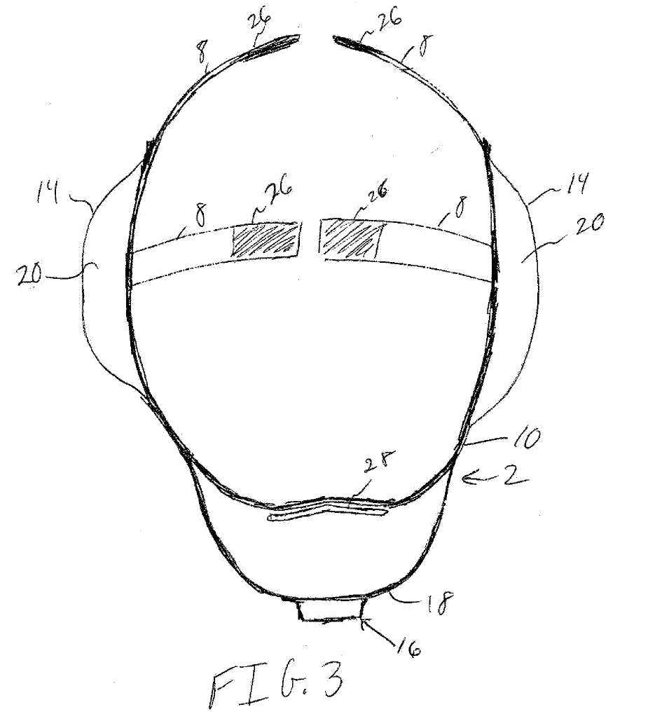

FIG. 4 depicts a dust mask 2 comprising a substantially unitary, substantially continuous body 10 wherein the dust mask 2 does not comprise an exhalation vent 16 and thus at least a portion of the body 10 of the dust mask 2 is porous and semi-permeable so that air and other desired elements can pass through the mask while particles and undesired elements cannot. Further, in FIG. 4, the dust mask 2 does not comprise straps as depicted in other figures but rather comprises a resilient ear hook 36, depicted in broken line as it is embedded in this embodiment within the earpiece 14 of the dust mask 2. The ear hook 36 is configured to wrap around the ear of the user and thereby hold the dust mask to the head of the user without the need for straps or additional structures. If desired, a given dust mask can have both ear hooks and straps, or other retaining devices as desired.

FIG. 5 depicts a rear view of only the earpieces 14 of a mask 2, the earpieces 14 connected by a unitary strap 38. In FIG. 5, the unitary strap 38 extends from the lower lateral regions 32 and upper lateral regions 34 of the earpieces 14 of the mask. In the embodiment depicted, the legs 40 of unitary strap 38 meet at a center 42 which in the embodiment depicted is a hollow circle. One advantage of this configuration is that the strap can be permanently affixed to the sides of the dust mask 2 so that a user need merely slip the dust mask over his/her head, without having to also attach straps to each other. Typically, the unitary strap 38 is made of an elastomeric material so that it can adapt to the size of an individual user's head.

FIG. 7 depicts a side view of a dust mask in place on the head of a user, the depiction in silhouette. In the embodiment shown, a first retaining strap extends between opposed, substantially lateral edges of the mask and substantially behind the head of the user, while a second retaining strap extends between or about the opposed, substantially upper lateral edges and extends substantially over the head of the user. This configuration can advantageously hold the dust mask 2 to the face of the user with improved comfort and conformation of the mask to the face of the user. The strap configuration in this Figure, comprising broad straps that both depart from an upper lateral area 34 of the body 10 of mask 2, as opposed to the bulk of the embodiments and aspects discussed herein, can be advantageously used in dust masks that do not reach all the way to the ears. Further, in this embodiment instead of or in addition to an exhalation vent in other figures, the mask 2 comprises a respirator 54.

FIG. 8 depicts in silhouette a mask 2 as discussed herein on the head of a user 4. In this embodiment, the upper contour 46 of the mask 2 has been specifically conformed to match the lower margin 48 of eye protection device 44 so that the lower margin 48 and the upper contour 46 match well together to provide improved protection for the user. In certain embodiments herein, the devices, systems, kits, etc., as well as methods of making and using, can comprise making, providing and/or using both the dust mask 2 with the correspondingly configured eye protection device 44. For example, a single kit may comprise multiple dust masks 2 along with one of the eye protection devices 44. Replacement dust masks 2 can then be sold or otherwise provided to the user to be used specifically in conjunction with a particular eyewear 44.

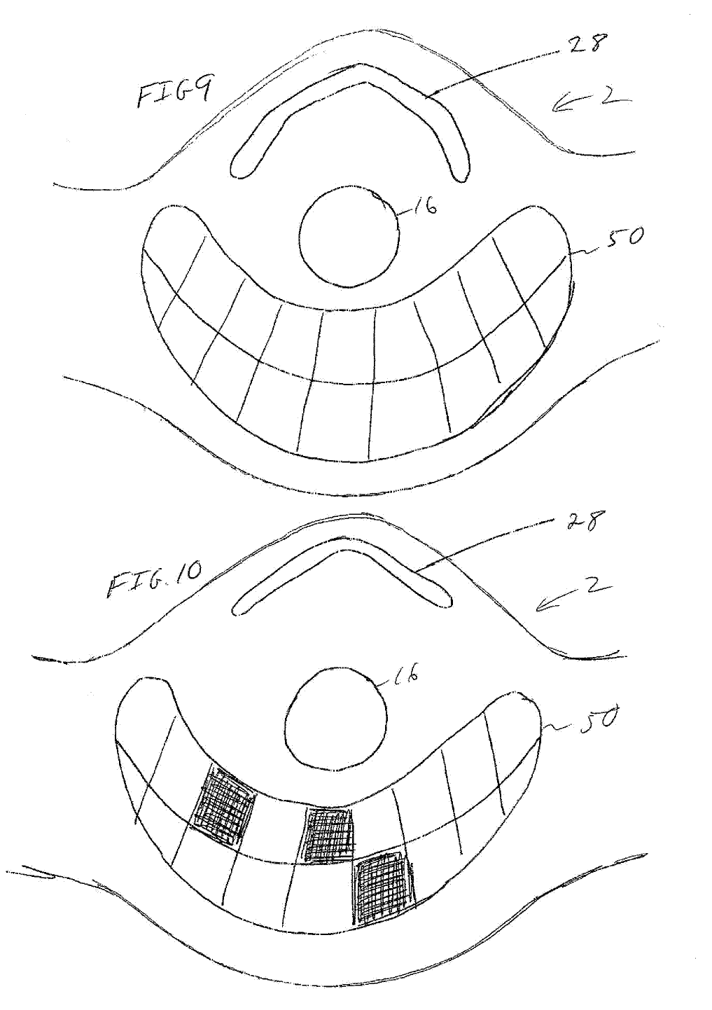

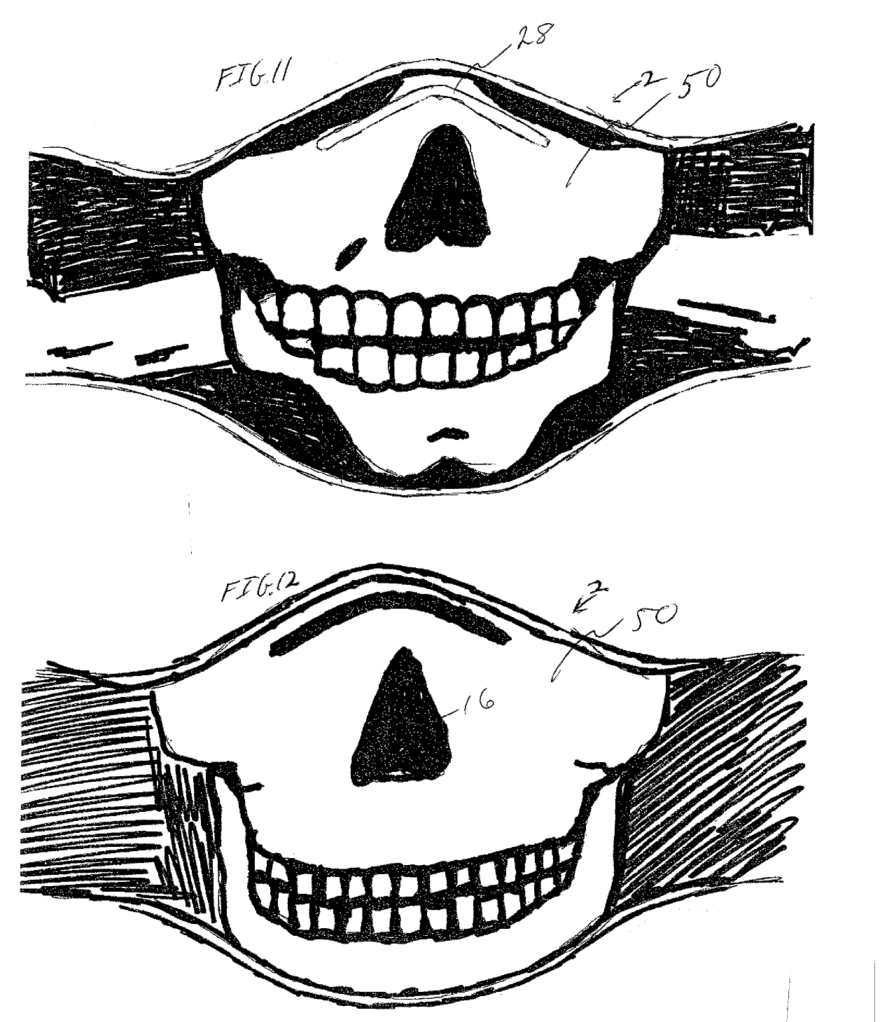

FIGS. 9-12 depict front plan views of various embodiments of the dust mask 2 herein further comprising a desired artistic design 50. In the embodiments shown, the artistic design takes advantage of the location and elements of the mask, for example, depicting teeth (either a complete set or a partial set), where the mouth of the user would be, putting an exhalation vent 16 where the nose of a user would be, etc. The design can also comprise, for example as shown in FIGS. 11 and 12, a skull as in FIG. 12 or a skull and crossbones as in FIG. 11, and may or may not comprise an exhalation element 16 where the black nose of the skull would be, thereby hiding the exhalation element if desired. The configuration of the artistic design 50 can be adapted to the intended environment for the dust mask 2. For example, in surgical situations or situations involving children where medical masks should be worn, a smile may help to place child patients at ease in an otherwise uncomfortable setting. The artistic designs depicted herein are merely exemplary; any desired artistic design can be implemented on the mask, including designs that do not have anything to do with the human face. Further, in certain embodiments of this aspect of the dust masks herein, the designs can be applied to dust masks that do not cover the ears.

Turning to a further discussion of the methods, devices, kits, etc., herein, the mask is typically substantially continuous from ear to ear and can be a simple unitary design (i.e., the mask is not only continuous from ear to ear but at least one ply of the layers of material making up the mask extends from ear to ear without a break). The masks typically comprise multiple layers of protective material plus one or more straps, resilient ear “hooks” or other retaining devices to hold the mask in place. The mask can also comprise, if desired, an exhalation vent and rigidity elements such as a nose bridge. The masks cover the mouth, nose and ears, protecting the user's hearing and keeping the ears clean without restricting the hearing ability (unless sound protection is also desired, in which case sound reducing materials and/or configurations can be provided). This mask can be made out of the same materials that other lightweight masks such as dust masks, surgical masks, etc., are made from, and are typically made of disposable materials. Also, the masks can be made of other materials such as activated carbon for use with different chemicals and particles.

The masks are typically designed to be easily put on and disposable and cost-effective, both to the manufacturer and the consumer. The masks are also typically very comfortable to wear. The straps need not go over (touching) the ear causing discomfort of the ears. The mask herein can be made in whatever materials are useful to keep certain unwanted elements out of a wearer's air passages, nose, mouth and lungs, or at least reduce the amount of such elements out of the wearer's air passages, etc. Also, the masks can protect the ears from the sun and may reduce ear infections and hearing loss.

Accordingly, in certain embodiments the devices, systems, methods, etc., discussed herein include masks configured to protect the ears, nose and mouth of a wearer. The masks can be suitably applied for any desired use such as dust protection, for example as found in an auto body shop, protection from hazardous chemicals, for example as found in the manufacture of fiberglass products and certain plastics, and in medical situations, or otherwise as desired. Some exemplary air-borne particulates for which the mask can provide protection include auto paint, asbestos (e.g., dust, fibers, particles), hazardous chemicals, pollen or fiberglass dust, blood splatter, and infectious organisms.

The specific materials used to make the mask can be selected from any desired set of materials appropriate for the given use. For example, where blood splatter may be of particular concern, then materials having pores small enough to prevent the transmission of blood from the outside to the user would be preferred. In situations where simple dust protection is the primary concern, then larger pores in the material can be used such that respiration is easier, and the need for an exhalation vent or other venting is reduced. The mask can be made of a single layer or multiple layers of a given material, such as a cloth, plastic, paper product, or other material as desired. Typically, the material comprising the bulk of the mask is itself porous, but if desired, the ability to inhale and exhale can be provided through one or more inhalation/exhalation vents (typically identified as an “exhalation vent” in the figures herewith).

Typically, the contours of the mask on the lower side of the mask are designed to run from behind the ears, along the jaw line of the user then to and/or under the chin, and along the upper line of the mask the edge of the mask typically runs from the ears, along the cheeks and then over the bridge of the nose. Typically, the mask is configured to cover both ears, the nose and the mouth, but in certain embodiments, for example where breathing must be conducted through a specialized oxygen source or respirator or the like, the mask may be configured to cover only the nose and the ears, or, conversely, the ears and mouth but not the nose. The mask does not include eye protection. However, if desired, the mask can advantageously be used in combination with separate eye protection, for example clear plastic eyewear such as used in operating rooms, welder's masks, or other eye protection as desired. In certain embodiments, the configuration of the upper edge of the mask near the eyes can be specifically conformed to match the lower margin of particular eye protection that the user will be wearing.

As noted previously, the construction of the mask is typically performed such that the mask is a substantially continuous mask going from one ear, over the nose and mouth, and on to cover the other ear. The mask may be held in place by any desired means, for example via rubber or elastic band(s) extending from one earpiece to the other, from hook-and-loop connector fabric (e.g., VELCRO®) strap(s) extending from one earpiece and the other, or by resilient elements configured to hook behind the user's ears and to hold the mask in place via mechanical interaction with the ears. The mask may contain one or more additional resilient elements, for example nosepieces, chin pieces, or ribbing or struts, and/or softening elements such as foam under the nosepiece, that can be configured to cause the masks to conform very closely to the user at desired points (for example, along the cheeks or jaw line), or to be held away from the user. Space between the mask and user can be advantageous, for example, around the mouth and nose, to provide a space between the user's mouth and nose as compared to the mask, and/or around the ears, for example to provide additional comfort so that contact of the ears with the mask is reduced, or to provide space to provide for additional materials such as sound deadening materials between the outer wall of the mask and the user's ears, or even large enough accommodate additional, separate devices such as ear muffs.

If desired, the mask can advantageously comprise one or more artistic designs, for example a skull and bones, a smile, or any other design as may be desired. In this specific embodiment, the systems, devices, methods, etc., herein can include lightweight dust masks and the like such as used by carpenters or car painters, which may or may not have the additional ear protection.

In some embodiments, the mask is provided as a single unitary piece of material that extends from one ear to the other, although if desired the mask can be made in pieces that are attached to each other; in others the mask. The mask is lightweight and should contain few elements where possible, both to reduce cost and to keep the weight down and therefore the comfort for the user up. Thus, the mask should fit snugly to the face, neck and ear region of the user substantially throughout the entirety of the mask. As desired, the mask can also include a liner around the edge formed for strength and/or improved fit relative to the user.

In certain embodiments, the straps holding the mask to the user's head are configured such that they comprise at least 2 straps, both departing the mask from or about the upper ear area. A first strap goes from the upper ear to substantially behind the head to the upper contour of the mask above the other ear, while a second retaining strap departs from substantially the same region and travels over the head of the user. If desired, one or both of the straps, typically the strap that travels behind the head, can be attached along a substantial distance along the upper contour (or even the ultimate upper edge) of the mask, thereby providing a substantially continuous retaining and holding force to the mask and reducing the effect of a possible “pinch-point” where the strap departs the mask from a single, small departure spot.

In certain embodiments, this strap configuration can be used in masks without ear protection, provided the lateral edge of the mask is sufficiently far back on the face to provide adequate purchase for the straps to be effective for the user.

The straps can be made in any desired fashion, for example (1), hook-and-fabric (e.g., VELCRO®) ends, tie cords (2), with a rubber circle or other “handle’ on the back of the nylon strip for grip (3), thicker VELCRO® straps (4), a pull-type strap that tightens up to fit.

The scope of the present devices, systems and methods, etc., includes both means plus function and step plus function concepts. However, the claims are not to be interpreted as indicating a “means plus function” relationship unless the word “means” is specifically recited in a claim, and are to be interpreted as indicating a “means plus function” relationship where the word “means” is specifically recited in a claim. Similarly, the claims are not to be interpreted as indicating a “step plus function” relationship unless the word “step” is specifically recited in a claim, and are to be interpreted as indicating a “step plus function” relationship where the word “step” is specifically recited in a claim.

From the foregoing, it will be appreciated that, although specific embodiments have been discussed herein for purposes of illustration, various modifications may be made without deviating from the spirit and scope of the discussion herein. Accordingly, the systems and methods, etc., include such modifications as well as all permutations and combinations of the subject matter set forth herein and are not limited except as by the appended claims or other claim having adequate support in the discussion herein.

1. A dust mask, the dust mask being lightweight, disposable and configured for a human face, the dust mask comprising a substantially continuous body sized and configured to extend from ear to ear and to cover all of and substantially only a mouth, nose and ears of a user, the mask further configured to transmit air inhaled by or exhaled by the user yet substantially completely block at least dust from reaching the nose, mouth and ears of the user.

2. The dust mask of claim 1 wherein the mask is further configured to substantially completely block at least one undesired air-borne contaminant in addition to dust.

3. The dust mask of claim 1 wherein the mask comprises a central portion covering the user's nose and mouth and two earpieces seamlessly attached to the central portion.

4. The dust mask of claim 1 wherein the mask further comprises a pliable, resilient nose bridge that can be adjusted to conform to the nose of the user.

5. The dust mask of claim 4 wherein the mask further comprises a softening element under the nose bridge.

6. The dust mask of claim 5 wherein the softening element is a foam piece.

7. The dust mask of claim 1 wherein the mask further comprises at least one retaining device configured to hold the mask in place on the user.

8. The dust mask of claim 7 wherein the retaining device comprises at least one strap extending from earpiece to earpiece of the mask or at least two resilient ear hooks configured to at least partially loop around the ears of the user to hold the mask in place via mechanical interaction with the ears.

9. The dust mask of claim 7 wherein the retaining device is the at least one strap extending from earpiece to earpiece and the strap extends from at least one of a top or back edge of each earpiece such that the strap does not contact the ears of the user.

10. The dust mask of claim 1 wherein the at least one strap comprises at least one of an elastic band or two opposed partial straps joining at mating male and female sides of a hook-and-loop connector fabric.

11. The dust mask of claim 1 wherein the mask comprises multiple layers of protective material extending from ear to ear.

12. The dust mask of claim 1 wherein the mask comprises at least one layer of protective material extending from ear to ear and at least one layer of protective material covering substantially only one or both of the mouth and nose.

13. The dust mask of claim 1 wherein the mask comprises at least one layer of protective material extending from ear to ear and at least one, non-continuous layer of protective material comprising at least two separate ear portions, each ear portion covering substantially only one ear.

14. The dust mask of claim 13 wherein the ear portions comprise at least one sound reducing material sufficient to protect the ears of a user from industrial levels of ambient sound.

15. The dust mask of claim 14 wherein the industrial level of ambient sound is at least one of an industrial spray painting machine, a cutting tool or an internal combustion engine.

16. The dust mask of claim 1 wherein the mask is an industrial dust mask configured to protect a user from at least one air-borne particulate.

17. The dust mask of claim 16 wherein the air-borne particulate is at least one of auto paint, asbestos, a hazardous chemical, pollen or fiberglass dust.

18. The dust mask of claim 1 wherein the mask is a medical mask configured to protect a user from at least one air-borne medically undesirable element.

19. The dust mask of claim 18 wherein the air-borne medically undesirable element is at least one of blood splatter, an infectious organism or a hazardous chemical.

20. The dust mask of claim 1 wherein the mask is made of at least one of cloth, plastic or paper products.

21. The dust mask of claim 1 wherein substantially all of the mask is porous.

22. The dust mask of claim 1 wherein the mask comprises at least one inhalation/exhalation vent.

23. The dust mask of claim 1 wherein a lower contour of the dust mask extends from behind the ears, along the jaw line of the user then to and/or under the chin.

24. The dust mask of claim 1 wherein an upper contour of the mask extends from the ears, along the cheeks and then over the bridge of the nose.

25. The dust mask of claim 1 wherein an upper contour of the mask is specifically conformed to match a lower margin of corresponding specific eye protection for the user.

26. The dust mask of claim 1 wherein the mask further comprises at least one of a resilient pliable chin piece, a resilient pliable ribbing, or a resilient pliable strut that can be configured to cause the periphery of the mask to at least one of conform very closely to the chin of a user or to create a desired, defined space between the interior of the mask and the user at least one desired point.

27. The dust mask of claim 1 wherein the mask is further configured to have respective bulges in each of the ear portions defining spaces between a user's ears and the respective ear portions.

28. The dust mask of claim 27 wherein the space is configured to fit at least one sound deadening material disposed between an outer wall of the mask and the user's ears.

29. The dust mask of claim 27 wherein the space is configured to fit an ear muff.

30. The dust mask of claim 1 wherein the mask comprises a space between the user's mouth and the interior of the mask sufficient to fit a respirator.

31. The dust mask of claim 1 wherein an outer wall of the mask further comprises at least one artistic design.

32. The dust mask of claim 31 wherein the artistic design is a skull and bones or a smile.

33. The dust mask of claim 1 wherein the mask further comprises a liner around substantially an entirety of an edge of the entire mask, the liner configured for improved strength and/or fit relative to the user.

34-77. (canceled)

78. A system comprising a plurality of dust masks according to claim 1 wherein the dust masks are stacked on each other such that the masks are nestled one inside another, and point of sale packaging substantially enclosing the stack of dust masks.

79. A system comprising a dust mask comprising the elements of claim 1 and a glasses or goggles configured for the human user.

80. The system of claim 79 wherein an upper contour of the dust mask and a lower contour of the glasses or goggles are configured to provide a substantially complementary fit to each other.

81-92. (canceled)
